(19) United States
(12) Patent Application Publication
(10) Pub. No.: US 2026/0297667 A1
(43) Pub. Date: Oct. 1, 2026

(54) Direct Template Indexing for Reuse of Spatial Indexing Substrates

(71) Applicant: The Broad Institute, Inc., Cambridge, MA (US)

(72) Inventors: Aziz Al'Khafaji, Cambridge, MA (US); Ghamdan Abdulqawi Al-Eryani, Cambridge, MA (US)

(73) Assignee: The Broad Institute, Inc., Cambridge, MA (US)

(21) Appl. No.: 19/576,444

(22) Filed: Mar. 24, 2026

Related U.S. Application Data
(60) Provisional application No. 63/777,325, filed on Mar. 25, 2025.

(51) Int. Cl. C12Q 1/6869; C12Q 1/6837

(52) U.S. Cl. CPC C12Q 1/6869; C12Q 1/6837

(57) ABSTRACT

Direct template indexing for reuse of spatial indexing substrates is described. Capture probes of a spatial indexing substrate having a plurality of spatial locations within a capture region are capped, each of the plurality of spatial locations having a unique spatial barcode for the capture probes therein. A biological sample is applied to the capture region. The applied biological sample is permeabilized to release nucleic acids from the biological sample. The released nucleic acids are annealed to the capture probes while preserving a spatial organization of the nucleic acids in the biological sample. The annealed nucleic acids are extended using the capture probes as templates, the extending incorporating the unique spatial barcode of an annealed capture probe into a given nucleic acid. The extended nucleic acids are eluted from the capture probes. The spatial indexing substrate is regenerated for reuse with a different biological sample.

### RELATED APPLICATION

This application claims priority to U.S. Provisional Patent Application Ser. No. 63/777,325, filed Mar. 25, 2025, entitled “Direct Template Indexing for Reuse of Spatial Indexing Substrates,” the entire disclosure of which is hereby incorporated by reference herein in its entirety.

### BACKGROUND

Conventional sequencing methods enable profiling of genetic and/or transcriptomic information. However, these approaches do not retain spatial context, which is a barrier to understanding the organization and functional interactions of cells. Spatial sequencing technologies have been developed to address this issue by combining sequencing with spatial localization. Spatial sequencing, for instance, enables gene expression to be interrogated in its native spatial context, providing information regarding how genes are expressed in different regions of a tissue. This information may provide insight into complex biological processes, disease mechanisms, and cellular interactions.

### SUMMARY

Direct template indexing for reuse of spatial indexing substrates is described. Capture probes of a spatial indexing substrate having a plurality of spatial locations within a capture region are capped, each of the plurality of spatial locations having a unique spatial barcode for the capture probes therein. A biological sample is applied to the capture region. The applied biological sample is permeabilized to release nucleic acids from the biological sample. The released nucleic acids are annealed to the capture probes while preserving a spatial organization of the nucleic acids in the biological sample. The annealed nucleic acids are extended using the capture probes as templates, the extending incorporating the unique spatial barcode of an annealed capture probe into a given nucleic acid. The extended nucleic acids are eluted from the capture probes. The spatial indexing substrate is regenerated for reuse with a different biological sample.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ direct template indexing for reuse of spatial indexing substrates as described herein.

FIG. 2 schematically illustrates an implementation of the spatial indexing substrate.

FIG. 3 shows an illustrative example process for direct template indexing for reuse of spatial indexing substrates as described herein.

FIG. 4 depicts an example implementation of a model system demonstrating direct template indexing for reuse of spatial indexing substrates as described herein.

FIG. 5 depicts an example procedure for direct template indexing for reuse of spatial indexing substrates as described herein.

FIG. 6 illustrates an example system including various components of an example device that can be implemented as any type of computing device as described and/or utilized with reference to FIGS. 1-5 to implement the techniques described herein.

### DETAILED DESCRIPTION

### Overview

As mentioned above, spatial sequencing technologies combine sequencing with spatial localization. Existing spatial sequencing methods typically include capturing RNA molecules (e.g., messenger RNA, or “mRNA”) from a tissue sample using spatially barcoded capture probes arranged on a spatial indexing substrate, such as a slide. Because of its use in spatial sequencing, the spatial indexing substrate may also be referred to as a spatial sequencing substrate. The spatially barcoded capture probes encode physical locations where the RNA molecules were captured on the slide, which corresponds to a location of origin of a given RNA molecule in the tissue sample. The capture is followed by reverse transcription, sequencing, and computational analysis to map gene expression patterns with respect to a physical location in the tissue sample. However, current spatial sequencing technologies face several challenges. For example, the high costs associated with specialized reagents and equipment used in spatial sequencing experiments hinder widespread adoption and large-scale studies.

Recent advances in sequencing technologies and throughputs have decreased sequencing costs, but the single-use nature of many spatial indexing substrates contributes to the high overall cost of experiments. For example, in currently available spatial sequencing techniques, the spatially barcoded capture probes act as primers for reverse transcribing mRNA molecules released from a permeabilized tissue sample. During this process, the mRNA serves as the template for complementary DNA (cDNA) synthesis. By way of example, each spatially barcoded capture probe includes a spatial barcode that identifies a location of the capture probe on the slide, and a reverse transcriptase enzyme extends the spatially barcoded capture probe by incorporating nucleotides that are complementary to the annealed mRNA. This generates cDNA that links the spatial barcode to genetic information of the mRNA. However, the spatially barcoded capture probe is irreversibly altered, as the newly synthesized cDNA is covalently attached to the capture probe. Thus, the spatial indexing substrate is consumed by processing a single tissue sample.

To overcome these issues, direct template indexing for reuse of spatial indexing substrates is disclosed herein. In accordance with the described techniques, a spatial indexing substrate is provided that includes capture probes with unique spatial barcodes arranged in a defined pattern across a surface of the spatial indexing substrate. The spatial indexing substrate may take various forms, non-limiting examples of which include planar surfaces, beads (or other particles), and the like. This versatility allows the technique to be applied not only to tissue samples, but also to cell suspensions or other samples. This enables, for example, single-cell RNA sequencing and other applications that benefit from spatial or cellular resolution. As used herein, a spatial barcode may refer to a nucleotide sequence that is specific to (e.g., unique to) a defined location on the spatial indexing substrate or to a specific bead, thereby encoding spatial and/or cellular information about the origin of nucleic acid molecules within a biological sample (e.g., a tissue sample) and enabling precise mapping of gene expression to tissue coordinates or individual cells.

The capture probes are capped with chain terminators at their 3′ ends to prevent extension and may include a capture sequence, such as a polythymidine sequence for capturing polyadenylated mRNA and/or a specific sequence designed for targeted nucleic acid capture. For example, the capture probes may include capture sequences complementary to the 3′ end of target RNAs, gene-specific sequences, consensus sequences for gene families, and/or other sequences configured to target one or more nucleic acids of interest. This design enables direct template indexing, where mRNA molecules from a tissue sample are extended using the capture probes as templates without altering the capture probes themselves. Accordingly, the spatial barcodes are incorporated into the mRNA without altering the capture probes or consuming the spatial indexing substrate. This approach allows for multiple uses of the same spatial indexing substrate, which may substantially reduce a cost per experiment as well as an amount of waste generated.

By way of example, when a tissue sample or cell suspension is applied to the spatial indexing substrate and permeabilized, released nucleic acid (e.g., mRNA) molecules anneal to the capture probes based on a particular capture sequence used (e.g., polythymidine for mRNA). A DNA polymerase enzyme that lacks reverse transcription activity (e.g., RNA-dependent polymerase activity) may be used to extend the nucleic acid molecule using the capture probe as a template. This incorporates the spatial barcode (e.g., a complement of the spatial barcode) into the extended nucleic acid molecule without altering the capture probe. The extended nucleic acid molecule may be eluted using a low ionic strength buffer and collected for further processing, as will be elaborated below. The spatial indexing substrate may be regenerated by washing with an alkaline solution to remove any residual nucleic acid, tissue matter, and/or cellular debris, allowing the spatial indexing substrate to be reused for spatial sequencing or single-cell sequencing of different samples without cross-contamination.

Following elution, the extended, spatially indexed nucleic acid undergoes reverse transcription to generate cDNA. The cDNA retains the spatial barcode information incorporated in the extended nucleic acid. In the context of tissues, the spatial barcode information maintains the spatial context (e.g., coordinate position) of each nucleic acid. In the context of single-cell sequencing, the spatial barcode information indicates a cell of origin. Accordingly, the cDNA is spatially indexed according to a location of the particular spatial barcode on the spatial indexing substrate and/or to a specific barcoded bead. The cDNA may be amplified and sequenced, producing spatially barcoded sequencing data. The spatially barcoded sequencing data, along with an image of the tissue sample on the spatial indexing substrate (as applicable), may be used to create a spatial gene expression map and/or to analyze single-cell transcriptomes. By way of example, spatial coordinates of a given sequencing read's nucleic acid molecule of origin in the tissue sample may be inferred based on the spatial barcode identified therein. As another example, the identity of the cell of origin may be inferred based on the spatial barcode identified therein. The spatial gene expression map may correlate gene expression levels with specific locations in the tissue and/or single cells, which may provide insights into the spatial organization of gene activity within the tissue sample or cell suspension.

By using the direct template indexing method described herein, spatial sequencing can be performed with significantly reduced costs and/or waste compared to conventional techniques with single-use substrates. Additionally, separating the spatial barcoding step from cDNA synthesis provides greater flexibility in optimizing reverse transcription and amplification protocols. The techniques described herein also maintain high spatial resolution and enable larger spatial sequencing studies to be performed that were previously cost-prohibitive.

In some aspects, the techniques described herein relate to a method for direct template indexing, including: capping capture probes of a spatial indexing substrate having a plurality of spatial locations within a capture region, each of the plurality of spatial locations having a unique spatial barcode for the capture probes therein; applying a biological sample to the capture region; permeabilizing the applied biological sample to release nucleic acids from the biological sample; annealing the released nucleic acids to the capture probes while preserving a spatial organization of the nucleic acids in the biological sample; extending the annealed nucleic acids using the capture probes as templates, the extending incorporating the unique spatial barcode of an annealed capture probe into a given nucleic acid; eluting the extended nucleic acids from the capture probes; and regenerating the spatial indexing substrate for reuse with a different biological sample.

In some aspects, the techniques described herein relate to a method, further including: generating complementary deoxyribonucleic acid (cDNA) from the eluted extended nucleic acids such that a given molecule of cDNA includes the unique spatial barcode of a corresponding molecule of the eluted extended nucleic acids; and sequencing the cDNA to generate spatially barcoded sequencing data.

In some aspects, the techniques described herein relate to a method, further including: generating a biological image by imaging the biological sample on the spatial indexing substrate prior to permeabilizing the biological sample; and generating a spatial gene expression map based on the spatially barcoded sequencing data and the biological image.

In some aspects, the techniques described herein relate to a method, wherein generating the spatial gene expression map includes: extracting spatial barcodes from the spatially barcoded sequencing data; mapping the extracted spatial barcodes to known locations of the capture region; and correlating gene expression data with morphological features identified in the biological image based on the extracted spatial barcodes.

In some aspects, the techniques described herein relate to a method, further including: applying the different biological sample to the capture region; permeabilizing the applied different biological sample to release different nucleic acids from the different biological sample; annealing the released different nucleic acids to the capture probes; extending the annealed different nucleic acids using the capture probes as templates, the extending incorporating the unique spatial barcode of a corresponding spatial location into a given different nucleic acid; eluting the extended different nucleic acids from the capture probes; and regenerating the spatial indexing substrate for reuse.

In some aspects, the techniques described herein relate to a method, wherein capping the capture probes includes adding a chain terminator to a 3′ end of each capture probe.

In some aspects, the techniques described herein relate to a method, wherein extending the annealed nucleic acids includes using a DNA polymerase enzyme that lacks RNA-dependent DNA polymerase activity.

In some aspects, the techniques described herein relate to a method, wherein eluting the extended nucleic acids includes washing the spatial indexing substrate with a low ionic strength buffer.

In some aspects, the techniques described herein relate to a method, wherein regenerating the spatial indexing substrate includes washing the spatial indexing substrate with an alkaline solution to remove residual nucleic acids and the permeabilized biological sample.

In some aspects, the techniques described herein relate to a system for spatial sequencing via direct template indexing, including: a spatial indexing substrate including: a substrate surface; a plurality of spatial locations on the substrate surface; and a plurality of capture probes attached to the substrate surface at each of the plurality of spatial locations, each of the plurality of capture probes capped with a chain terminator on a 3′ end and having a spatial barcode that is specific to a corresponding spatial location; and a sequencing data processor executing instructions stored in a non-transitory computer-readable storage medium to perform operations including: receiving spatially indexed sequencing data generated from complementary deoxyribonucleic acid (cDNA) of spatially indexed messenger ribonucleic acid (mRNA), each molecule of the spatially indexed mRNA including an mRNA molecule released from a tissue sample onto the spatial indexing substrate and extended to include the spatial barcode using one of the plurality of capture probes as a template; and generating a spatial gene expression map based on the spatially indexed sequencing data and an image of the tissue sample on the spatial indexing substrate.

In some aspects, the techniques described herein relate to a system, wherein each of the plurality of capture probes includes, from 5′ to 3′: an anchor sequence that attaches to the substrate surface; an amplification target sequence designed for primer binding during reverse transcription of the spatially indexed mRNA to the cDNA; the spatial barcode; a unique molecular identifier (UMI) sequence; a capture sequence configured to anneal to a target nucleic acid sequence; and the chain terminator.

In some aspects, the techniques described herein relate to a system, wherein the capture sequence is a polythymidine sequence, and the target nucleic acid sequence is a polyadenylation sequence.

In some aspects, the techniques described herein relate to a system, wherein the spatial indexing substrate is used with a plurality of different tissue samples during separate spatial sequencing experiments via a regeneration process performed after the spatially indexed mRNA is eluted from the plurality of capture probes.

In some aspects, the techniques described herein relate to a system, wherein the mRNA molecule released from the tissue sample onto the spatial indexing substrate is extended to include the spatial barcode using a polymerase enzyme that lacks an RNA-dependent DNA polymerase activity.

In some aspects, the techniques described herein relate to a method for spatial sequencing via direct template indexing, including: applying a tissue sample to a capture region of a spatial indexing substrate, the spatial indexing substrate having a plurality of spatial locations, each spatial location having capture probes with a spatial barcode that is specific to the spatial location and a 3′ chain terminator; permeabilizing the applied tissue sample to release messenger ribonucleic acid (mRNA) from the applied tissue sample to the capture probes while preserving a spatial organization of the mRNA in the applied tissue sample; annealing the released mRNA to the capture probes; extending the annealed mRNA using a polymerase enzyme that lacks RNA-dependent deoxyribonucleic acid (DNA) polymerase activity and using the capture probes as templates, the extending producing spatially indexed mRNA encoding the spatial barcode that is specific to the spatial location of an annealed capture probe; eluting the spatially indexed mRNA from the capture probes; generating complementary DNA (cDNA) from the spatially indexed mRNA such that a given molecule of cDNA includes the spatial barcode of a corresponding molecule of the spatially indexed mRNA; sequencing the cDNA to generate spatially barcoded sequencing data; and regenerating the spatial indexing substrate by washing the spatial indexing substrate with an alkaline solution.

In some aspects, the techniques described herein relate to a method, wherein the 3′ chain terminator is a dideoxynucleotide.

In some aspects, the techniques described herein relate to a method, further including generating a tissue image by imaging the applied tissue sample on the spatial indexing substrate prior to permeabilizing the applied tissue sample.

In some aspects, the techniques described herein relate to a method, further including generating a spatial gene expression map based on the spatially barcoded sequencing data and the tissue image.

In some aspects, the techniques described herein relate to a method, wherein each capture probe further includes a unique molecular identifier (UMI) sequence that is incorporated into the spatially indexed mRNA during the extending.

In some aspects, the techniques described herein relate to a method, further including using the UMI sequence to identify and remove duplicate reads in the spatially barcoded sequencing data.

In the following discussion, an example environment is first described that may employ the techniques described herein. Example implementation details and procedures are then described which may be performed in the example environment as well as other environments. Consequently, performance of the example procedures is not limited to the example environment, and the example environment is not limited to performance of the example procedures.

### Example Environment

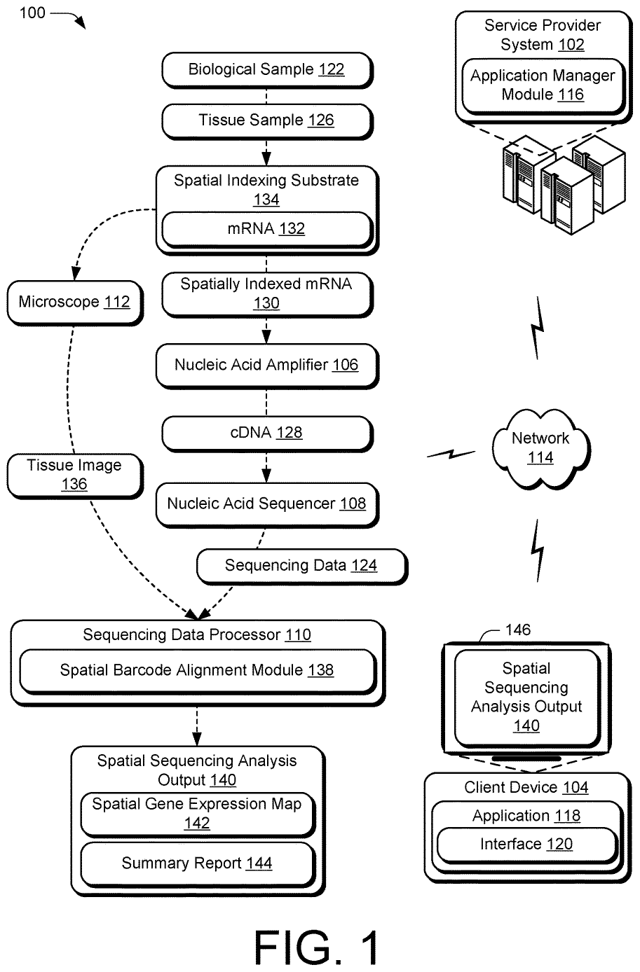

FIG. 1 is an illustration of an environment 100 in an example implementation that is operable to employ direct template indexing for reuse of spatial indexing substrates as described herein. The illustrated environment 100 includes a service provider system 102, a client device 104, a nucleic acid amplifier 106, a nucleic acid sequencer 108, a sequencing data processor 110, and a microscope 112. One or more or each of the service provider system 102, the client device 104, the nucleic acid sequencer 108, the sequencing data processor 110, and the microscope 112 may be communicatively coupled, one to another, via a network 114. The network 114 may enable wired and/or wireless electronic communication, for example. Although the sequencing data processor 110 is illustrated as separate from the service provider system 102, the client device 104, and the nucleic acid sequencer 108, this functionality may be incorporated as part of the service provider system 102, the client device 104, and/or the nucleic acid sequencer 108, further divided among other entities, and so forth. By way of example, an entirety of or portions of the functionality of the sequencing data processor 110 may be incorporated as part of the nucleic acid sequencer 108 and/or the client device 104. Additionally or alternatively, an entirety of or portions of the client device 104 may be incorporated as part of the nucleic acid sequencer 108 and/or the sequencing data processor 110. Moreover, in at least one variation, the nucleic acid amplifier 106, the nucleic acid sequencer 108, and/or the microscope 112 is not communicatively coupled to the network 114.

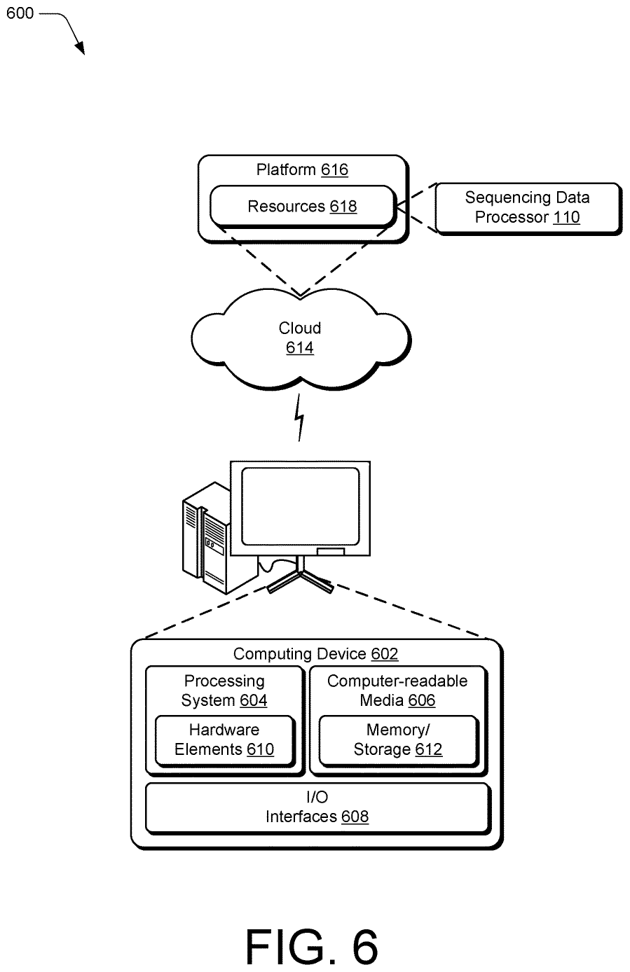

Computing devices that are usable to implement the service provider system 102, the client device 104, and the sequencing data processor 110 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full-resource devices with substantial memory and processing resources (e.g., personal computers, consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 6.

The service provider system 102 is illustrated as including an application manager module 116 that is representative of functionality to provide access to the sequencing data processor 110 to a user of the client device 104 via the network 114. The application manager module 116, for instance, may expose content or functionality of the sequencing data processor 110 that is accessible via the network 114 by an application 118 of the client device 104. The application 118 may be configured as a network-enabled application, a browser, a native application, and so on, that exchanges data with the service provider system 102 via the network 114. The data can be employed by the application 118 to enable the user of the client device 104 to communicate with the service provider system 102, such as to receive application updates and features when the service provider system 102 provides functionality to manage the application 118.

In the context of the described techniques, the application 118 includes functionality to analyze data generated by a spatial sequencing event. In the illustrated example, the application 118 includes an interface 120 that is implemented at least partially in hardware of the client device 104 for facilitating communication between the client device 104 and the sequencing data processor 110. By way of example, the interface 120 includes functionality to receive inputs to the sequencing data processor 110 from the client device 104 (e.g., from a user of the client device 104) and output information, data, and so forth from the sequencing data processor 110 to the client device 104, as will be further elaborated herein.

The spatial sequencing event includes determining an order of nucleotides (e.g., adenine, thymine or uracil, cytosine, and guanine) in a sample of nucleic acids, such as nucleic acids derived from a biological sample 122, while preserving spatial information. The order of nucleotides is referred to herein as a “sequence,” which may be determined from sequencing data 124. The nucleotides are also referred to as “bases.” The spatial sequencing event will be described herein with respect to the sequencing of complementary deoxyribonucleic acid (cDNA) derived from messenger ribonucleic acid (mRNA) transcripts while maintaining their spatial context within a tissue sample 126. However, in at least one variation, the tissue sample 126 may be a fresh frozen, methanol fixed, or cryopreserved tissue sample, for instance, although the techniques described herein may be adapted for tissues preserved via other methods (e.g., formalin-fixed, paraffin-embedded tissues). The tissue sample 126 may be a section or slice of the biological sample 122, for instance. The biological sample 122, and thus the tissue sample 126, may be obtained from a subject (e.g., an individual, such as a patient, or another type of organism) and/or a culture.

The nucleic acid sequencer 108 is configured to produce the sequencing data 124, which are analyzed by the sequencing data processor 110 to determine the order of nucleotides in the nucleic acid sample and their spatial locations. In at least one implementation, the sequencing data 124 comprise a text-based file format, such as FASTQ files that store both nucleotide sequence information and quality scores for the bases in a sequencing read. In variations, the sequencing data 124 may comprise another type of file format. The nucleic acid sequencer 108 may use one or more sequencing techniques to produce the sequencing data 124, e.g., “sequencing reads” or “reads.” By way of example, the nucleic acid sequencer 108 may use a short-read sequencing technique that produces sequence fragments typically ranging from approximately 10 bases to approximately 1000 bases and more typically from approximately 50 bases to approximately 500 bases. Sequence fragments produced via short-read sequencing techniques are also referred to as “short reads.” Alternatively, the nucleic acid sequencer 108 may use a long-read sequencing technique that produces sequence fragments typically ranging from approximately 1000 bases to 1,000,000 bases and more typically from 5000 bases to 600,000 bases in length. Sequence fragments produced via long-read sequencing techniques are also referred to as “long reads.” In at least one variation, the nucleic acid sequencer 108 may use another type of sequencing technique that produces sequencing reads of an intermediate length, such as approximately 1500 bases (e.g., a length in a range between 1000 and 5000 bases).

Regardless of the sequencing technique, in the illustrated example environment 100, the nucleic acid sequencer 108 produces the sequencing data 124 for cDNA 128 that is synthesized from spatially indexed mRNA 130 derived from the tissue sample 126. The spatially indexed mRNA 130 comprises spatial barcode-labeled versions of mRNA 132 transcribed for gene expression in the tissue sample 126. By way of example, each molecule of the spatially indexed mRNA 130 includes a spatial barcode indicating the original location of the corresponding mRNA 132 within the tissue, as labeled using a spatial indexing substrate 134. It is to be appreciated that although the spatial indexing will be described with respect to the mRNA 132 by way of illustration, other targeted nucleic acid capture is also contemplated, as further elaborated herein.

By way of example, the spatially indexed mRNA 130 is generated by annealing the mRNA 132 released from the tissue sample 126 onto the spatial indexing substrate 134. In at least one implementation, the spatial indexing substrate 134 comprises a surface having spatially arranged capture probes, each of the spatially arranged capture probes including a spatial barcode and a capture sequence. The spatial indexing substrate 134 may include various types of physical substrates. By way of example, the capture probes may be attached to a single surface of the spatial indexing substrate 134, such as a generally planar surface. Alternatively, the spatial indexing substrate 134 may include a plurality of beads, with each bead having a different (e.g., unique) spatial barcode. In at least one other variation, the spatial indexing substrate 134 may include a microfluidic device or another three-dimensional structure that can be functionalized with the spatially barcoded capture probes. The spatial barcode is specific to a location (or bead) of a given capture probe on the spatial indexing substrate 134, whereas the capture sequence may not be specific to a particular location or bead, at least in some examples. By way of example, the capture sequence may be configured to drive hybridization and templated extension of a particular nucleic acid molecule (e.g., having a targeted sequence) or type of nucleic acid molecule of interest (e.g., mRNA in general).

In at least one implementation, the capture sequence includes deoxythymidine (dT) nucleotides (also referred to as an “oligo dT” or “poly(T) sequence”). The poly(T) sequence is configured to anneal to a polyadenylation (e.g., “poly(A)”) sequence, such as the 3′ poly(A) tail of the mRNA 132 released from the tissue sample 126, through complementary binding (e.g., base pairing through hydrogen bonding, where A pairs with T/U and C pairs with G). In at least one variation, the capture sequence includes a sequence complementary to the 3′ end of a target nucleic acid in order to selectively anneal nucleic acids having a particular sequence of interest. Alternatively, or in addition, a first portion of the capture probes may include a first capture sequence targeting a first nucleic acid (or type of nucleic acid), a second portion of the capture probes may include a second capture sequence targeting a second nucleic acid (or type of nucleic acid), and so forth. By way of example, a given capture sequence may target a gene-specific sequence, a gene family-specific consensus sequence, a non-coding RNA sequence, a viral or bacterial sequence (e.g., for evaluating infected tissues), a specific splice variant or isoform, and the like.

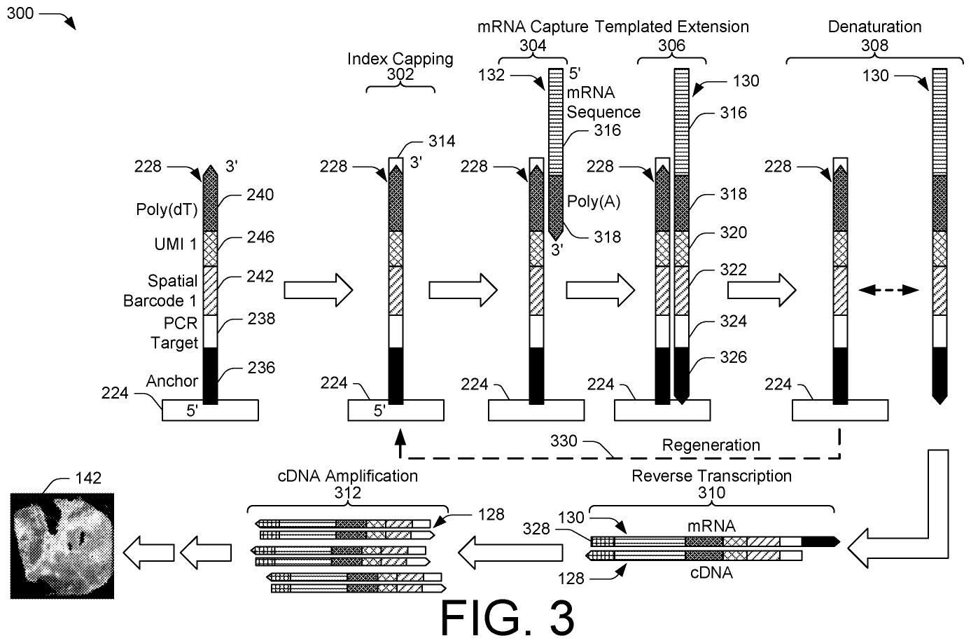

During the indexing process, the mRNA 132 molecules (or other target, such as described above) from the tissue sample 126 are captured by the probes while preserving their original spatial context, and the spatial barcode of the spatially indexed mRNA 130 indicates the original location of the mRNA 132 within the tissue sample 126. As will be elaborated herein, e.g., with respect to FIG. 3, the spatial indexing substrate 134 is not consumed during the spatial indexing process, allowing for multiple rounds of spatial indexing on the same surface.

When annealed to the poly(A) sequences of respective molecules of the mRNA 132, the capture probes of the spatial indexing substrate 134 may serve as a template for extending the mRNA 132 by a DNA polymerase enzyme to produce the spatially indexed mRNA 130. By way of example, the DNA polymerase enzyme may add nucleotide triphosphates that are complementary to the annealed capture probe to the mRNA 132. In this process, the spatial barcode is physically appended to the mRNA 132 without altering the capture probes themselves. This is accomplished using a polymerase that lacks RNA-dependent DNA polymerase activity. Such polymerases can extend from an RNA primer but cannot perform reverse transcription. As an example, the DNA polymerase may be a Klenow fragment (e.g., a DNA polymerase I enzyme having its exonuclease domain removed). The lack of an exonuclease domain may ensure that the capture probes are not altered or degraded. As a result, the spatial indexing substrate 134 acts as a template and is not altered or consumed during the indexing process.

In at least one implementation, the nucleotide triphosphates are deoxyribonucleotide triphosphates. Accordingly, the mRNA 132 may be extended to include a DNA sequence that is complementary to the annealed capture probe, and the spatially indexed mRNA 130 may be an mRNA/DNA hybrid molecule.

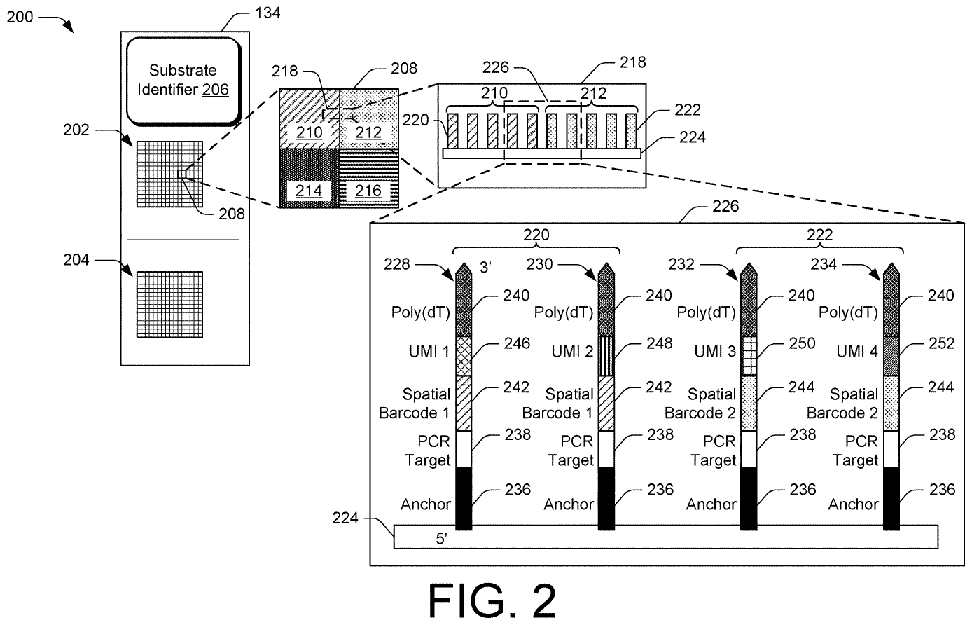

The capture probes on the spatial indexing substrate 134 may further include additional sequences. These sequences may include, for example, a unique molecular identifier (UMI) that uniquely labels cDNA derived from a particular mRNA 132 molecule, thus enabling an RNA molecule of origin of a given sequencing read in the sequencing data 124 to be identified during a downstream analysis and one or more target sequences for subsequent reverse transcription and/or amplification. Moreover, the capture probes may include a chain terminator (e.g., a blocking group) on their 3′ ends to prevent extension of the capture probes. Additionally, or alternatively, the additional sequences may enable the cDNA 128 to bind to a solid support or surface, such as the surface of a sequencing flow cell or a substrate (e.g., a magnetic bead or another type of affinity purification substrate) used for purification. Additional details regarding an example structure of the capture probe will be described herein, e.g., with respect to FIG. 2.

Following the templated indexing process, the spatially indexed mRNA 130 may be eluted from the spatial indexing substrate 134 using low ionic strength buffer washes. The spatially indexed mRNA 130 is collected and used to synthesize the cDNA 128, such as via reverse transcription and amplification steps. This approach allows the spatial indexing substrate 134 to be reused for many different tissue samples 126. For example, the spatial indexing substrate 134 may be washed and reused at least five times (e.g., for five different labeling processes for five different tissue samples 126), which is not possible using the currently available spatial indexing techniques.

Accordingly, the cDNA 128 comprises a plurality of cDNA molecules that are complementary to respective mRNA molecules of the spatially indexed mRNA 130, maintaining the spatial barcode information encoded therein. This approach enables the sequencing data 124 to retain spatial information about gene expression within the tissue sample 126, providing information regarding the gene expression of cells in their native context.

In at least one implementation, the cDNA 128 is synthesized from the spatially indexed mRNA 130 at the nucleic acid amplifier 106. The nucleic acid amplifier 106 is an instrument that facilitates cDNA synthesis through a reverse transcription reaction. The nucleic acid amplifier 106 may further facilitate second-strand synthesis and/or amplification of the cDNA 128 via one or more reactions. By way of example, the nucleic acid amplifier 106 may be a thermal cycler that is configured to cycle through different temperature stages, which allow for the denaturation of nucleic acid (e.g., by disrupting secondary structures of the spatially indexed mRNA 130 or the cDNA 128), annealing of primers (e.g., short oligonucleotides that bind to a target portion of the spatially indexed mRNA 130 or the cDNA 128), extension of new, complementary DNA strands using an enzyme (e.g., a DNA polymerase enzyme), and inactivation of the enzyme to terminate the reaction.

The reactions carried out in the nucleic acid amplifier 106, such as the extension of the mRNA 132 and/or the reverse transcription reaction, may be performed in one or more rounds, also referred to herein as “reaction cycles.” A reaction cycle, for instance, may include a denaturation step followed by an annealing step, which is followed by an extension step. To facilitate this, the nucleic acid amplifier 106 may include a thermal block or heating/cooling element to regulate temperature, a programmable interface to set reaction cycle parameters (e.g., temperature and time), and heating/cooling mechanisms to rapidly transition between the different temperature stages. The nucleic acid amplifier 106 may further include a heated lid to prevent condensation of the samples during the reaction.

The cDNA 128 synthesized from the spatially indexed mRNA 130 may include a particular order or structure of predefined sequences (e.g., a spatial barcode, adapter sequence(s), a poly(T) sequence or poly(A) sequence, etc.). Accordingly, the sequencing data processor 110 is configured to receive the sequencing data 124 from the nucleic acid sequencer 108 and determine sequences of the cDNA 128, and thus the sequences of the originating mRNA, therefrom based at least in part on the structure of the read and the sequences found therein. In at least one implementation, the sequences of the cDNA 128 and/or the originating mRNA may be included in a spatial sequencing analysis output 140.

In at least one implementation, the spatial indexing substrate 134 further functions as a slide for imaging the tissue sample 126 via the microscope 112. The microscope 112, for instance, may be used to capture a tissue image 136 of the tissue sample 126 on the spatial indexing substrate 134. The tissue image 136 may provide visual information about the spatial organization and morphology of the tissue sample 126. By way of example, the microscope 112 may be a brightfield microscope (e.g., when the tissue sample 126 is stained with hematoxylin and eosin) and/or a fluorescence microscope (e.g., when immunofluorescence is used for staining the tissue sample 126). The microscope 112, for instance, may have a high enough resolution to distinguish histological structures. In at least one implementation, the tissue image 136 is obtained before the tissue sample 126 is permeabilized to release the mRNA 132 onto the spatial indexing substrate 134.

The sequencing data processor 110 may include a spatial barcode alignment module 138, which is representative of functionality to process both the sequencing data 124 and the tissue image 136 to generate a spatial sequencing analysis output 140. By way of example, the spatial barcode alignment module 138 may perform image processing of the tissue image 136. The image processing may include tiling and/or stitching (e.g., when multiple smaller fields of view of the tissue sample 126 are captured by the microscope 112); color, brightness, and/or contrast adjustment(s); and/or image segmentation.

By way of example, the spatial barcode alignment module 138 may use one or more image processing techniques to analyze the tissue image 136 and identify distinct regions or features within the tissue sample 126. In at least one implementation, the spatial barcode alignment module 138 performs image segmentation to delineate cell boundaries, identify specific cell types based on morphological characteristics, and/or indicate tissue structures/anatomical landmarks.

In parallel or sequentially, the spatial barcode alignment module 138 may process the sequencing data 124, which contains information about gene expression levels along with the spatial barcodes that indicate the original locations of the mRNA 132 molecules within the tissue sample 126. By way of example, the spatial barcode alignment module 138 may perform quality filtering, barcode extraction, and/or deduplication as a part of processing the sequencing data 124. Quality filtering, for instance, may include removing low-quality reads (e.g., based on quality scores associated with the respective reads in the sequencing data 124). As another example, when the spatial indexing substrate 134 includes UMIs, one read for a given UMI may be selected for further analysis in order to provide accurate transcript quantification. Reads that have the same UMI are assumed to have the same mRNA 132 of origin in the tissue sample 126. Duplicates may arise during amplification of the cDNA 128 prior to sequencing, for instance. The spatial barcode alignment module 138 may further perform transcriptome alignment, where the reads of the sequencing data 124 are mapped to a reference genome and/or transcriptome.

The barcode extraction process performed by the spatial barcode alignment module 138 may include parsing the sequencing reads of the sequencing data 124 to locate the barcode sequence based on its known position within the read structure. This position may be determined based on the design of the capture probes of the spatial indexing substrate 134. As elaborated herein, these spatial barcodes correspond to specific locations on the spatial indexing substrate 134, allowing the spatial barcode alignment module 138 to map the sequencing reads back to an original spatial location of the mRNA 132 in the tissue sample 126.

In at least one implementation, the spatial barcode alignment module 138 may reference a predefined list of spatial barcodes, which may be referred to as a barcode whitelist. This whitelist may further indicate the known spatial position (e.g., X and Y coordinates) of each spatial barcode on the spatial indexing substrate 134. The spatial barcode alignment module 138 may compare extracted barcodes against this whitelist to correct for potential sequencing errors and/or to filter out reads with invalid barcodes.

After extraction, the spatial barcode alignment module 138 may use the spatial barcodes to group sequencing reads by their spatial origin within the tissue sample 126. This grouping allows the spatial barcode alignment module 138 to generate a spatial gene expression map 142, which may be included in the spatial sequencing analysis output 140. The spatial gene expression map 142 may provide a visual representation of gene expression patterns across the tissue sample 126, correlating with the tissue image 136 captured by the microscope 112. For example, the spatial gene expression map 142 may be visualized as a heatmap and/or a cluster analysis. The spatial gene expression map 142 may indicate how gene expression varies across different regions of the tissue, which may reveal spatial patterns of gene activity, identify cell types based on their expression profiles, and/or highlight areas of differential gene expression associated with specific tissue structures and/or pathological features. The spatial gene expression map 142 may offer insights into complex biological processes, disease mechanisms, and cellular interactions that may not be apparent from sequencing data or imaging alone.

In one or more implementations, the spatial barcode alignment module 138 may employ machine learning algorithms or statistical methods to enhance the accuracy of the alignment between the sequencing data and the tissue image. For example, an image registration algorithm may be used to correct for potential distortions or variations in the tissue during processing, which may increase an accuracy of mapping the gene expression data to the spatial context of the tissue sample 126.

In at least one implementation, the spatial sequencing analysis output 140 further includes a summary report 144. The summary report 144 may indicate quality control metrics, spatial mapping statistics, and/or gene expression insights, for instance. By way of example, the quality control metrics may include factors such as read depth, sequencing coverage, and/or alignment scores. The spatial mapping statistics may indicate how effectively the sequencing reads were mapped back to their original locations in the tissue sample 126. By way of example, the spatial mapping statistics may include one or more or each of a total number of spatial locations (e.g., a spot on the spatial indexing substrate 134 having a particular spatial barcode) detected in the sequencing data 124, an average number of genes detected per spatial location, and a spatial registration alignment score. The gene expression insights may indicate spatially variable genes, gene expression patterns across different tissue regions, and/or correlations between gene expression and specific tissue structures or cell types. The summary report 144, for instance, may provide a comprehensive yet concise overview of the spatial transcriptomics data. It is to be appreciated that additional or different information may be included in the spatial sequencing analysis output 140, including the spatial gene expression map 142 and/or the summary report 144, without departing from the spirit or scope of the described techniques.

The client device 104 is shown displaying, via a display device 146, the spatial sequencing analysis output 140. It is to be appreciated that the sequencing data 124 and/or the spatial sequencing analysis output 140 may be also stored in a memory of the sequencing data processor 110 and/or the client device 104 for subsequent access.

In this way, the direct template indexing techniques described herein enable high spatial sequencing resolution while enabling multiple uses of the spatial indexing substrate 134. The reusability of the spatial indexing substrate 134 substantially reduces the cost and waste per experiment, making spatial transcriptomics more accessible. Moreover, the separation of the spatial barcoding step from the cDNA synthesis step also offers greater flexibility in optimization of reverse transcription and amplification protocols without being constrained by the spatial indexing substrate 134.

### Direct Template Indexing for Reuse of Spatial Indexing Substrates

FIG. 2 schematically illustrates an implementation 200 of the spatial indexing substrate 134 introduced in FIG. 1. It is to be appreciated that the implementation 200 is a simplified example, and the relative lengths of the various sequence portions and components are not to scale.

The spatial indexing substrate 134 may include multiple capture regions. In the example of the implementation 200, the spatial indexing substrate 134 includes a first capture region 202 and a second capture region 204, although the spatial indexing substrate 134 may include any number of capture regions. By way of example, in variations, the spatial indexing substrate 134 may include one, three, or more capture regions. The spatial indexing substrate 134 may take various forms, including, but not limited to, planar surfaces, beads or other particles, microfluidic devices, and/or other structured surfaces. The spatial indexing substrate 134 may further include a substrate identifier 206, which may be used to uniquely identify the spatial indexing substrate 134. The substrate identifier 206 may include an alphanumeric code, a scannable barcode, a QR code, a radio-frequency identification tag, or another type of identifier. The substrate identifier 206 may encode information such as a serial number, manufacturing details, and/or specifications of the spatial indexing substrate 134.

A first magnified view 208 of the first capture region 202 shows four spatial locations: a first spatial location 210, a second spatial location 212, a third spatial location 214, and a fourth spatial location 216. These spatial locations may represent distinct areas on the substrate surface 224 where capture probes are arranged. The spatial locations may be arranged in a regular pattern across the substrate surface 224, with each location including capture probes having a unique spatial barcode with respect to the other locations. This arrangement may allow for high-resolution spatial mapping of captured mRNA molecules to their original positions within the tissue sample 126 (not shown in FIG. 2). By way of example, each spatial location may have an edge length of approximately 2 micrometers. In variations, the edge length is another measurement, such as a measurement within a range between 1 micrometer and 5 micrometers. When implemented as barcoded beads/particles, each bead/particle may represent a unique spatial location having a unique spatial barcode, enabling applications such as reusable single-cell RNA sequencing.

Although the implementation 200 shows the spatial locations as having a square shape, variations are possible. For example, the spatial locations may be circular, rectangular, hexagonal, or another type of polygon. However, a square shape may enable dense, continuous packing of spatially barcoded capture probes while enabling simpler image registration due to the alignment of edges and corners in a regular grid pattern. This regular grid pattern may facilitate accurate mapping of the sequencing reads to original positions of the mRNA 132 on the spatial indexing substrate 134, and thus to the tissue image 136. Moreover, in at least one variation, the spatial locations may be spaced apart from each other such that there is a larger distance between the capture probes of adjacent spatial locations than between the capture probes within the same spatial location.

A second magnified view 218 provides a closer look at the spatial locations, depicting a first capture probe set 220 within the first spatial location 210 and a second capture probe set 222 within the second spatial location 212. For instance, the second magnified view 218 is a side view, in contrast to the top view of the first magnified view 208. The first capture probe set 220 and the second capture probe set 222, as well as other capture probe sets for the other spatial locations, are arranged on a substrate surface 224, which may be glass, a polymer (e.g., plastic), or another material that may be selected based on its optical properties (e.g., for imaging the tissue sample 126 with the microscope 112), chemical stability, and ease of functionalization for attaching the capture probes. The first capture probe set 220 and the second capture probe set 222 differ based on the spatial barcode included therein, as further elaborated herein.

A third magnified view 226 provides an example structure of the capture probes, showing four individual probe molecules to highlight the similarities and differences between the different probe molecules in different spatial locations. The third magnified view 226 shows a first probe molecule 228, a second probe molecule 230, a third probe molecule 232, and a fourth probe molecule 234 anchored to the substrate surface 224 via their 5′ ends. The first probe molecule 228 and the second probe molecule 230 are part of the first capture probe set 220 of the first spatial location 210, and the third probe molecule 232 and the fourth probe molecule 234 are part of the second capture probe set 222 of the second spatial location 212. It is to be appreciated that although the implementation 200 shows equal spacing between the first probe molecule 228, the second probe molecule 230, the third probe molecule 232, and the fourth probe molecule 234, variations are possible. For instance, a distance between the second probe molecule 230 and the third probe molecule 232 may be greater than that between the second probe molecule 230 and the first probe molecule 228, such as when the spatial locations are spaced apart with respect to each other.

Each probe molecule includes multiple sequence portions arranged in a specific order from 5′ to 3′. In the implementation 200, the probe molecules include, from 5′ to 3′, an anchor sequence 236, a polymerase chain reaction (PCR) target sequence 238, a spatial barcode sequence, a unique molecular identifier (UMI) sequence, and a capture sequence 240 (e.g., a polythymidine sequence, or “poly(dT),” in this example, where the dT denotes deoxythymidine). The anchor sequence 236 may attach the probe molecule to the substrate surface 224. The PCR target sequence 238 may serve as a universal primer binding site for downstream reverse transcription and/or amplification. The spatial barcode sequence may encode location information of the probe on the substrate and is unique to the particular spatial location of the capture region. The UMI sequence may allow for identification of individual captured mRNA molecules. The capture sequence 240 may be designed to anneal to a specific target nucleic acid or type of nucleic acid, e.g., through hydrogen bonding. In the implementation 200 shown in FIG. 2, the capture sequence 240 is a poly(T) sequence configured to anneal to the poly(A) tail of mRNA molecules, enabling capture (e.g., reversible binding) of the mRNA 132 released from the tissue sample 126.

The individual capture probe molecules may be differentiated by their barcode sequences and UMI sequences. By way of example, the first probe molecule 228 and the second probe molecule 230 include a first barcode sequence 242 (e.g., “spatial barcode 1”), while the third probe molecule 232 and the fourth probe molecule 234 include a second barcode sequence 244 (e.g., “spatial barcode 2”). The first barcode sequence 242 is specific to the first capture probe set 220 and the first spatial location 210 (e.g., every capture probe molecule in the first capture probe set 220, which is located within the first spatial location 210 and not any other spatial location, includes the first barcode sequence 242). Similarly, the second barcode sequence 244 is specific to the second capture probe set 222 and the second spatial location 212. Accordingly, the first barcode sequence 242 and the second barcode sequence 244 encode spatial information that indicates the position of the capture probe within the first capture region 202.

The spatial barcode sequences, including the first barcode sequence 242 and the second barcode sequence 244, include known sequences ranging from about ten to about fifty nucleotides in length. The first barcode sequence 242 and the second barcode sequence 244, as well as other spatial barcode sequences included in the spatial indexing substrate 134, may have the same length or may vary in length. The first barcode sequence 242 includes at least one nucleotide difference from the second barcode sequence 244, as well as other spatial barcode sequences of the spatial indexing substrate 134. In at least one implementation, the spatial barcode sequences may differ from each other by at least two to five nucleotides to account for sequencing errors and ensure robust differentiation between spatial locations.

The UMI sequence differentiates one capture probe molecule from another, alone or in combination with the spatial barcode sequence. In the implementation 200, the first probe molecule 228 has a first identifier sequence 246 (e.g., “UMI 1”), the second probe molecule 230 has a second identifier sequence 248 (e.g., “UMI 2”), the third probe molecule 232 has a third identifier sequence 250 (e.g., “UMI 3”), and the fourth probe molecule 234 has a fourth identifier sequence 252 (e.g., “UMI 4”). The UMI sequences may typically range from about eight to about twenty nucleotides in length and may differ from each other by at least two to five nucleotides to account for sequencing errors and ensure robust differentiation between capture probe molecules.

The UMI sequences may enable precise quantification of individual mRNA molecules. By way of example, a first mRNA molecule captured by the first probe molecule 228 may be labeled with complements of the first identifier sequence 246 and the first barcode sequence 242, as will be further described below with respect to FIG. 3. A second mRNA molecule captured by the second probe molecule 230 may be labeled with complements of the second identifier sequence 248 and the first barcode sequence 242. As such, sequencing reads derived from the first mRNA molecule are able to be differentiated from those derived from the second mRNA molecule, as well as other mRNA molecules captured by the first capture probe set 220, based on the first identifier sequence 246.

In at least one variation, the combination of the UMI and the spatial barcode may uniquely identify each mRNA molecule of origin, enabling the same UMI sequences to be used in the different capture locations. By way of example, the UMI sequences within the first spatial location 210 may be unique with respect to each other, but not with respect to those in the second spatial location 212, the third spatial location 214, and/or the fourth spatial location 216. As an illustrative, non-limiting example, the third probe molecule 232 may include the first identifier sequence 246 (e.g., rather than the third identifier sequence 250). The third probe molecule 232 may capture a third mRNA molecule. Reads corresponding to the third mRNA molecule may be distinguished from those corresponding to the first mRNA molecule based on the combination of the first identifier sequence 246 and the second barcode sequence 244. In contrast, reads corresponding to the first mRNA molecule may be identified based on the combination of the first identifier sequence 246 and the first barcode sequence 242.

Overall, the arrangement and composition of the capture probe molecules on the substrate surface 224 may allow for templated indexing of mRNA molecules from tissue samples without consuming or altering the capture probe molecules, as will be further elaborated below.

FIG. 3 shows an illustrative example process 300 for direct template indexing for reuse of spatial indexing substrates as described herein. It is to be appreciated that the process 300 is a simplified example, and the relative lengths of the various sequence portions are not to scale. Moreover, for illustrative clarity, particular sequence portions are not labeled in every section of the figure. Sequence portions that are shaded the same are meant to denote the same sequence or a complement thereof.

The process 300 includes index capping 302, mRNA capture 304, templated extension 306, denaturation 308, reverse transcription 310, and cDNA amplification 312. It is to be appreciated that the first probe molecule 228 is given as an illustrative example, and the process 300 may occur similarly with other probe molecules (e.g., the second probe molecule 230, the third probe molecule 232, the fourth probe molecule 234, and any other probe molecule of the first capture region 202 and/or the second capture region 204).

During the index capping 302, a chain terminator 314 is added to the 3′ end of the first probe molecule 228. The chain terminator 314 may include a single chain-terminating base, which may be added to the first probe molecule 228 via a terminal transferase enzyme, which functions to add nucleotides to the 3′ end of a DNA molecule without a template. The chain terminator 314 may be part of a dideoxynucleotide triphosphate (ddNTP) mix, for example. The ddNTPs lack a 3′-hydroxyl group, which is involved in the formation of a phosphodiester bond between two nucleotides, thus preventing further extension of the DNA chain (e.g., the first probe molecule 228) beyond the chain terminator 314. That is, an additional nucleotide is unable to bond with the chain terminator 314. Accordingly, the index capping 302 prevents extension of the first probe molecule 228 during subsequent steps of the process 300. Following the index capping 302, the spatial indexing substrate 134 may be washed to remove unused ddNTPs and terminal transferase enzyme.

The mRNA capture 304 shows the mRNA 132 hybridizing to the first probe molecule 228. For example, the tissue sample 126 may be positioned on the first capture region 202, imaged via the microscope 112 of FIG. 1, and permeabilized (e.g., using a permeabilization enzyme) to release the mRNA 132. Upon permeabilization, the mRNA 132 anneals to the capture probes of the first capture region 202 in a spatially preserved manner, meaning that the original spatial organization and relative positions of the mRNA 132 within the tissue sample 126 are maintained.

The mRNA 132 includes an mRNA sequence 316 at the 5′ end and a poly(A) sequence 318 at the 3′ end. The mRNA sequence 316 is the coding region of the mRNA that includes the genetic information (e.g., as transcribed from a gene) to be translated into a protein. The mRNA 132 is shown as one molecule in FIG. 3, although it is to be appreciated that other molecules of the mRNA 132 may hybridize with other probes within the first capture region 202. The mRNA 132 anneals to the first probe molecule 228 via complementary base pairing of the poly(A) sequence 318 to the capture sequence 240.

During the templated extension 306, a DNA polymerase enzyme extends the mRNA 132 in the 3′ direction by adding nucleotides, e.g., deoxyribose nucleotide triphosphates (dNTPs), complementary to the first probe molecule 228. The templated extension 306 may be performed in the nucleic acid amplifier 106, for example. In at least one implementation, the DNA polymerase enzyme is Klenow fragment (exo-) polymerase, which can extend from an RNA primer but cannot perform reverse transcription because it lacks RNA-dependent DNA polymerase activity. The Klenow fragment (exo-) polymerase also cannot remove the chain terminator 314. The nucleic acid amplifier 106 may be programmed according to desired temperature conditions for the Klenow fragment activity.

By way of example, the templated extension 306 results in nucleotides complementary to the sequences on the first probe molecule 228 extending from the mRNA 132 until the DNA polymerase enzyme reaches the 5′ end of the first probe molecule 228. The nucleic acid amplifier 106 may be programmed to control the duration of the templated extension 306, allowing sufficient time for complete extension while minimizing non-specific reactions. This results in the spatially indexed mRNA 130 including, from 5′ to 3′, the mRNA sequence 316, the poly(A) sequence 318, and newly synthesized complementary sequences including a first identifier sequence complement 320 (which is complementary to the first identifier sequence 246), a first barcode sequence complement 322 (which is complementary to the first barcode sequence 242), a PCR target sequence complement 324 (which is complementary to the PCR target sequence 238), and an anchor sequence complement 326 (which is complementary to the anchor sequence 236).

The denaturation 308 separates the spatially indexed mRNA 130 from the first probe molecule 228 on the substrate surface 224. The denaturation 308 may include washing the substrate with a low ionic strength buffer, which weakens the hydrogen bonds between the complementary base pairs, allowing for the collection of the spatially indexed mRNA 130. In at least one implementation, the spatial indexing substrate 134 is serially washed with the low ionic strength buffer, and the serial washes may be collected separately and/or pooled. By way of example, through multiple washing steps with the low ionic strength buffer, the hydrogen bond forces driving hybridization of the mRNA 132 to the first probe molecule 228 are weakened, allowing for dissociation. The denaturation 308 may further include applying mild heat (e.g., in a temperature range from 32-37° C.), such as by using the nucleic acid amplifier 106 as a heating block.

In the reverse transcription 310, the spatially indexed mRNA 130 is used as a template to synthesize the cDNA 128. In at least one implementation, a primer including the PCR target sequence 238 anneals to the PCR target sequence complement 324 of the spatially indexed mRNA 130. A reverse transcriptase enzyme extends this primer by incorporating dNTPs, creating the first strand of the cDNA 128. As a part of this, when the reverse transcriptase enzyme reaches the 5′ end of the spatially indexed mRNA 130, it adds a few non-templated nucleotides, typically cytosines, to the 3′ end of the newly synthesized cDNA strand. This creates a 3′ overhang on the cDNA. A template switching oligonucleotide (TSO) 328, which has a 3′ end complementary to these added cytosines, hybridizes to the 3′ overhang of the cDNA 128. The reverse transcriptase switches templates and continues synthesis using the TSO 328 as a template, thereby incorporating the complement of the TSO 328 into the 3′ end of the cDNA 128.

The resulting cDNA 128 includes, from 5′ to 3′, the PCR target sequence 238, the first barcode sequence 242, the first identifier sequence 246, the sequence 240, a sequence complementary to the mRNA sequence 316, and a sequence complementary to the TSO 328. The sequence complementary to the TSO 328 at the 3′ end of the cDNA 128 and the PCR target sequence 238 at the 5′ end may serve as priming sites for second-strand synthesis and/or the cDNA amplification 312.

In the cDNA amplification 312, multiple copies of the cDNA 128 are generated. This amplification may include PCR or another amplification technique, which may be performed using the nucleic acid amplifier 106. By way of example, the cDNA amplification 312 may utilize primers targeting the PCR target sequence 238 and the TSO 328, or complements thereof, to amplify an entire length of the cDNA 128. Accordingly, the amplified cDNA 128 includes the spatial indexing information of the first barcode sequence 242, the transcript tracking information of the first identifier sequence 246, and the gene expression information of the mRNA sequence 316. The cDNA amplification 312 may duplicate both (e.g., complementary) strands of the cDNA 128. The amplified cDNA 128 can then be used for library construction and sequencing (e.g., at the nucleic acid sequencer 108), and the resulting sequencing data 124 may be used to generate the spatial gene expression map 142.

Moreover, because the first probe molecule 228 acts as a template and is not altered or consumed during the mRNA capture 304, the templated extension 306, and the denaturation 308, the first probe molecule 228 (and other probe molecules of the spatial indexing substrate 134) can be reused for a different tissue sample 126. The different tissue sample 126 may be a different portion (e.g., slice) of a same tissue of origin or a different tissue of origin. The different tissue of origin may be from a different type of tissue from a same organism or a same type of tissue from a different organism. That is, a tissue sample that is subsequently processed on the spatial indexing substrate 134 may be independent of a previously processed tissue sample 126.

Accordingly, the process 300 includes a regeneration 330. The regeneration 330 includes a series of washes to remove residual RNA while keeping the DNA-based probes, including the first probe molecule 228, intact. In at least one implementation, the stringent washes may utilize a wash buffer including an appropriate concentration of an alkaline solution that removes RNA molecules without damaging or altering the covalently attached DNA capture probes on the substrate surface 224. As a non-limiting example, the wash buffer used in the regeneration 330 is 0.08 molar (M) potassium hydroxide (KOH) solution, although other molarities and/or buffer compositions are possible. The regeneration 330 may include multiple washing steps, with the number of washes and specific conditions (such as temperature and duration) selected to maximize RNA removal efficiency.

Following the washing steps, the spatial indexing substrate 134 may be equilibrated with a neutral buffer to restore conditions suitable for subsequent use. The regenerated spatial indexing substrate 134 can then be employed for capturing and indexing mRNA from a different tissue sample 126, as described above. Accordingly, the regeneration 330 may enable multiple rounds (e.g., at least five rounds) of spatial sequencing experiments to be performed using the same spatial indexing substrate 134. This reduces the costs and waste associated with spatial sequencing experiments compared to currently available techniques, which use the mRNA 132 as a template for extending the capture probe rather than using the capture probe as a template for extending the mRNA 132.

Having discussed example details of the techniques for direct template indexing for reuse of spatial indexing substrates, consider now an example to illustrate usage of the techniques.

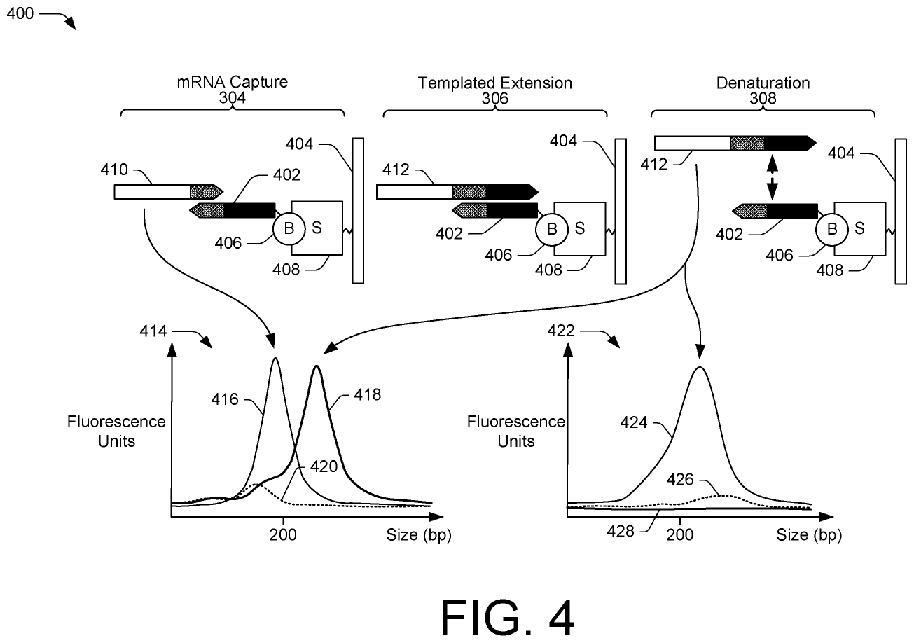

FIG. 4 depicts an example implementation 400 of a model system demonstrating direct template indexing for reuse of spatial indexing substrates as described herein. The implementation 400 illustrates the mRNA capture 304, the templated extension 306, and the denaturation 308 steps introduced in FIG. 3 in a simplified model system.

In the implementation 400, a capture oligonucleotide 402 is attached to a surface 404 via a linkage between a biotin 406 attached to the 5′ end of the capture oligonucleotide 402 and a streptavidin 408 attached to the surface 404. A strong non-covalent interaction between the biotin 406 and the streptavidin 408 securely attaches the capture oligonucleotide 402 to the surface 404, allowing the capture oligonucleotide 402 to remain fixed on the surface 404 throughout the direct template indexing process. In variations, other high-affinity binding pairs may be used to link the capture oligonucleotide 402 to the surface 404. Alternatively, or in addition, covalent attachment methods such as click chemistry, UV crosslinking, or other types of attachment methods may be used to immobilize the capture oligonucleotide on the surface 404. In still other variations, rather than a planar shape for the surface 404, the capture oligonucleotide 402 may be affixed to a bead or other type of surface.

During the mRNA capture 304, a polyadenylated RNA molecule 410 hybridizes to the capture oligonucleotide 402 immobilized on the surface 404, e.g., via a 3′ poly(A) tail of the polyadenylated RNA molecule 410 binding to a 5′ poly(T) sequence of the capture oligonucleotide 402. In the example of the implementation shown in FIG. 4, the polyadenylated RNA molecule 410 and capture oligonucleotide 402 are synthetic, simplified substrates used to demonstrate the chemistry of the direct template indexing techniques described herein. The polyadenylated RNA molecule 410 may measure approximately 200 base pairs (bp) in length, while the capture oligonucleotide 402 may measure approximately 100 bp in length, for instance.

The templated extension 306 shows the extension of the polyadenylated RNA molecule 410 to form an extended RNA/DNA hybrid 412. This extension occurs using the capture oligonucleotide 402 as a template. In the denaturation 308, the extended RNA/DNA hybrid 412 is separated from the capture oligonucleotide 402. By way of example, the extended RNA/DNA hybrid 412 is eluted from the capture oligonucleotide 402 via a series of washes with a low ionic strength buffer.

A size comparison graph 414 shows fluorescence units (vertical axis) versus size in base pairs (horizontal axis) and includes a polyadenylated RNA plot 416, an extended RNA plot 418, and a capture oligonucleotide plot 420. The polyadenylated RNA plot 416 corresponds to the polyadenylated RNA molecule 410, displaying its initial size distribution and relative abundance before template extension. The capture oligonucleotide plot 420 represents the capture oligonucleotide 402, indicating the size and relative quantity of the immobilized capture oligonucleotides on the surface. The extended RNA plot 418 corresponds to the extended RNA/DNA hybrid 412, demonstrating the size distribution and relative abundance of the DNA hybrid 412 after the templated extension 306 and the denaturation 308. This plot shows an increase in size of the extended RNA/DNA hybrid 412 compared to the polyadenylated RNA molecule 410, providing evidence of successful extension using the capture oligonucleotide 402 as a template.

An elution graph 422 provides quantitative evidence of the efficiency and completeness of release of the extended RNA/DNA hybrid 412 from the capture oligonucleotide 402 during the denaturation 308 step. The elution graph 422 shows fluorescence units (vertical axis) against size (horizontal axis) for three sequential elutions. A first elution plot 424 shows the highest fluorescence intensity, indicating a majority of the extended RNA/DNA hybrid 412 is released in an initial wash. A second elution plot 426 and a third elution plot 428 exhibit decreasing fluorescence intensities, demonstrating diminishing amounts of the extended RNA/DNA hybrid 412 released in subsequent washes. This pattern demonstrates that the denaturation 308 effectively separates the extended RNA/DNA hybrid 412 from the capture oligonucleotide 402, while also indicating that three washes may be sufficient for capturing the extended RNA/DNA hybrid 412.

Having discussed example details of the techniques for direct template indexing for reuse of spatial indexing substrates, consider now an example procedure to illustrate additional aspects of the techniques.

### Example Procedure

This section describes an example procedure for direct template indexing for reuse of spatial indexing substrates in one or more implementations. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks. In one or more implementations, at least a portion of the procedure is performed by a suitably configured device, such as the sequencing data processor 110 of FIG. 1, by executing instructions stored in a non-transitory computer-readable storage medium.

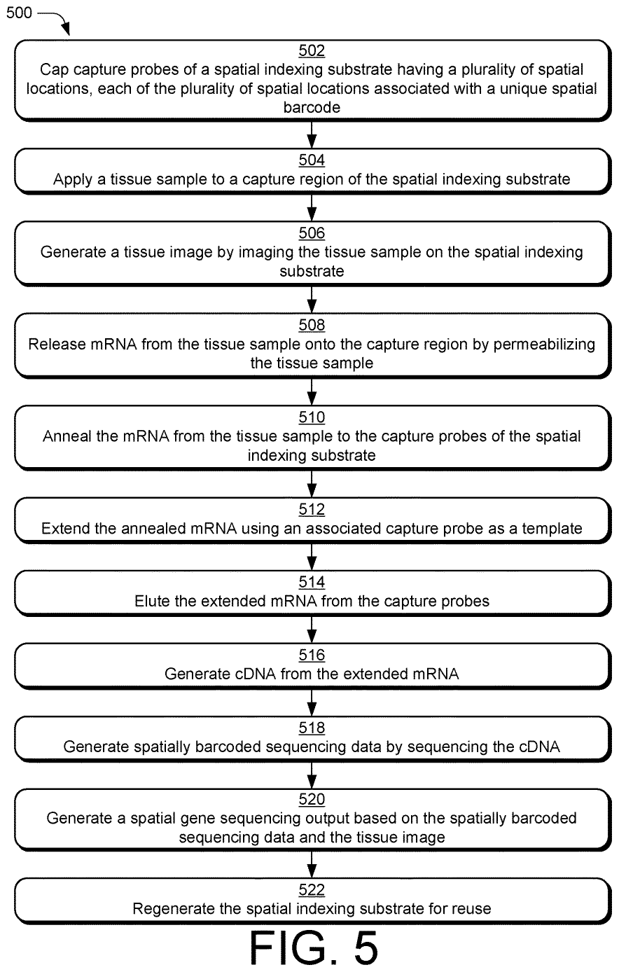

FIG. 5 depicts an example procedure 500 for direct template indexing for reuse of spatial indexing substrates as described herein.

Capture probes of a spatial indexing substrate having a plurality of spatial locations are capped, each of the plurality of spatial locations associated with a unique spatial barcode (block 502). By way of example, the capture probes may be capped using the index capping 302 described with reference to FIG. 3. The capping may include adding a chain terminator 314 to the 3′ end of each capture probe. The chain terminator 314 may be a single chain-terminating base added via a terminal transferase enzyme, preventing further extension of the capture probes. The capping process enables the spatial indexing substrate 134 to be reused for multiple tissue samples 126 without altering the capture probes during the indexing process. It is to be appreciated that after the index capping 302 is performed for a given spatial indexing substrate 134, block 502 may be omitted prior to processing a subsequent tissue sample 126.

A tissue sample is applied to a capture region of the spatial indexing substrate (block 504). By way of example, the tissue sample 126 may be positioned on the first capture region 202 of the reusable spatial indexing substrate 134 or the second capture region 204 of the reusable spatial indexing substrate 134, such as described in relation to FIG. 2. The tissue sample 126 may be derived from the biological sample 122, which may be obtained from a subject (e.g., an individual, such as a patient, or another type of organism) and/or a culture. The tissue sample 126 may be prepared by sectioning the biological sample 122 to create thin slices, typically ranging from about 5 to 20 micrometers in thickness, using a cryostat or microtome, for example. These thin sections may allow for better penetration of reagents and increased imaging resolution. The tissue sample 126 may be placed onto the capture region of the spatial indexing substrate 134. In at least one variation, instead of a tissue sample, a cell suspension may be applied to the spatial indexing substrate 134, such as when the spatial indexing substrate 134 comprises barcoded beads for single-cell RNA sequencing applications.

A tissue image is generated by imaging the tissue sample on the spatial indexing substrate (block 506). By way of example, the microscope 112 may be used to capture the tissue image 136 of the tissue sample 126 on the reusable spatial indexing substrate 134. The microscope 112 may be a brightfield microscope when the tissue sample 126 is stained with hematoxylin and eosin. As another example, the microscope 112 may be a fluorescence microscope, such as when immunofluorescence is used for staining the tissue sample 126. The microscope 112 may have a high enough resolution to distinguish histological structures. The tissue image 136 may provide visual information about the spatial organization and morphology of the tissue sample 126, which can be later correlated with gene expression data.

mRNA is released from the tissue sample onto the capture region by permeabilizing the tissue sample (block 508). By way of example, permeabilizing the tissue sample 126 releases the mRNA 132 onto the reusable spatial indexing substrate 134. The permeabilization process may be achieved through various methods, such as chemical treatment (e.g., with a detergent), enzymatic digestion (e.g., using proteinase K), and/or physical methods (e.g., freeze-thaw cycles). Permeabilization disrupts cell membranes and allows intracellular contents, including mRNA 132, to diffuse out of the cells while maintaining the overall tissue structure to preserve spatial information. In at least one variation, the permeabilization step may release other types of nucleic acids, such as non-coding RNAs or specific RNA targets, depending on the design of the capture probes.

The mRNA from the tissue sample is annealed to the capture probes of the spatial indexing substrate (block 510). By way of example, the annealing process corresponds to the mRNA capture 304 illustrated in FIG. 3, where the mRNA 132 anneals to the first probe molecule 228 via complementary base pairing of the poly(A) sequence 318 to the capture sequence 240 (e.g., the poly(T) sequence). In at least one variation, the annealing may occur between a specific target sequence of the nucleic acid and a complementary sequence on the capture probes, rather than through poly(A)/poly(T) interactions. This variation allows for targeted capture of specific genes, gene families, or non-coding RNAs, for instance. Each capture probe on the reusable spatial indexing substrate 134 includes a spatial barcode corresponding to its specific location on the substrate surface 224. For instance, capture probes at the first spatial location 210 include the first barcode sequence 242, while those at the second spatial location 212 include the second barcode sequence 244. When the mRNA 132 molecules anneal to these spatially arranged capture probes, they become associated with location information, allowing their original positions within the tissue sample 126 to be inferred during subsequent analysis.

The annealed mRNA is extended using an associated capture probe as a template (block 512). By way of example, the extension process corresponds to the templated extension 306 shown in FIG. 3, where a DNA polymerase enzyme extends the mRNA 132 in the 3′ direction by adding nucleotides complementary to the associated capture probe. The templated extension 306 results in the creation of spatially indexed mRNA 130, which includes the spatial barcode information. The spatially indexed mRNA 130 may include, from 5′ to 3′, the mRNA sequence 316, the poly(A) sequence 318 (and/or another targeted sequence), and newly synthesized complementary sequences to the associated capture probe, including a UMI sequence and the spatial barcode sequence of the associated capture probe. The spatial barcode sequence provides spatial context for each mRNA molecule captured from the tissue sample 126, and the UMI sequence enables amplicons and sequencing data derived from a single mRNA molecule of origin to be identified during downstream processing. The complementary sequences may further include an amplification target sequence (e.g., the PCR target sequence 238), which may facilitate downstream amplification and sequencing processes.

It is to be appreciated that although referred to as “spatially indexed mRNA” or “extended mRNA,” the extension product may be an mRNA/DNA hybrid. That is, the mRNA 132 may be extended with deoxynucleotide triphosphates, which are found in DNA, rather than ribonucleotide triphosphates, which are found in RNA. Moreover, in at least one variation with respect to single-cell applications, the extension may occur within individual droplets or wells, each containing a single cell and a spatially barcoded bead or particle as the spatial indexing substrate 134.

The extended mRNA is eluted from the capture probes (block 514). By way of example, the elution includes separating the spatially indexed mRNA 130 from the capture probes and the substrate surface 224, such as described with respect to the denaturation 308. The spatially indexed mRNA 130 may be eluted by washing the spatial indexing substrate 134 using a low ionic strength buffer, which weakens the hydrogen bonds between the complementary base pairs of the spatially indexed mRNA 130 and the capture probes. The elution buffer may be applied in multiple rounds to ensure sufficient release of the spatially indexed mRNA 130. In at least one implementation, mild heat (e.g., in a temperature range from 32-37° C.) may be applied to further facilitate the separation. The elution buffer is collected in order to collect the spatially indexed mRNA 130. The denaturation 308 leaves the capture probes intact on the substrate surface 224, preserving their structure and functionality. The eluted and collected spatially indexed mRNA 130 may be purified and quantified.

cDNA is generated from the extended mRNA (block 516). By way of example, the cDNA may be generated via the reverse transcription 310 and the cDNA amplification 312 described with respect to FIG. 3. In at least one implementation, a primer for the PCR target sequence 238 anneals to the spatially indexed mRNA 130, and a reverse transcriptase enzyme extends the primer using the spatially indexed mRNA 130 as a template, thus creating the first cDNA strand. Template switching occurs when the reverse transcriptase enzyme reaches the 5′ end of the spatially indexed mRNA 130 and incorporates the TSO 328. The TSO 328 may serve as a universal priming site for subsequent amplification (e.g., in a polymerase chain reaction), for example. The resulting cDNA 128 includes the UMI sequence and the spatial barcode sequence, or complements thereof. In at least one implementation, the cDNA amplification 312 may further introduce adapters for sequencing. In at least one variation where specific nucleic acids are targeted, the reverse transcription and amplification steps may be optimized for the particular targets, such as by using gene-specific primers or targeted amplification strategies.

Spatially barcoded sequencing data are generated by sequencing the cDNA (block 518). By way of example, the nucleic acid sequencer 108 produces the sequencing data 124, which may include reads spanning a length (e.g., an entire length) of a given molecule of the cDNA 128, including the spatial barcode sequence, a gene-specific sequence, and any additional incorporated elements (e.g., the UMI sequence). The sequencing data 124 also include quality scores for each base. The nucleic acid sequencer 108 may use short-read or long-read sequencing techniques. Depending on the sequencing method and the cDNA 128 length, a single read may encompass the full cDNA sequence, or multiple overlapping reads may be used to reconstruct the complete sequence.

A spatial gene sequencing output is generated based on the spatially barcoded sequencing data and the tissue image (block 520). By way of example, the spatial barcode alignment module 138 may process the sequencing data 124 and the tissue image 136. The spatial barcode alignment module 138 may perform quality filtering, spatial barcode sequence extraction, and alignment of sequencing reads to a reference genome or transcriptome. The spatial barcode alignment module 138 may use the spatial barcode sequences to map each read to its original position in the tissue sample 126. Additionally, the spatial barcode alignment module 138 may utilize the UMI sequences to identify and remove reads corresponding to PCR duplicates, enabling more accurate quantification of gene expression levels. This process creates a spatial gene expression map 142 that visualizes gene expression patterns across different regions of the tissue, correlating molecular data with morphological features observable in the tissue image 136.

In at least one variation where single-cell RNA sequencing is performed, cell-specific transcriptome profiles may be generated rather than a spatial gene expression map. The spatial sequencing analysis output 140 may include clustering cells based on their gene expression profiles and identifying cell types or states within the sample, for example.

The spatial indexing substrate is regenerated for reuse (block 522). By way of example, the regeneration 330 may include performing a series of washes to remove residual RNA from the spatial indexing substrate 134 while preserving the DNA-based capture probes on the substrate surface 224. The wash buffer may include an alkaline solution, such as a potassium hydroxide solution, that removes RNA molecules without damaging or altering the attached DNA-based capture probes. Multiple washing steps may be performed to maximize RNA removal. The regeneration 330 enables the spatial indexing substrate 134 to be used for multiple rounds of spatial transcriptomics experiments with different tissue samples 126, thus decreasing the costs and physical waste associated with spatial sequencing.

By way of example, the procedure 500 may be repeated with a second, different tissue sample. In at least some implementations, after the spatial indexing substrate 134 is initially capped (e.g., block 502 of the procedure 500 is performed for a given spatial indexing substrate), the index capping 302 of block 502 may be omitted.

Having described an example procedure in accordance with one or more implementations, consider now an example system and device that can be utilized to implement the various techniques described herein.

### Example System and Device

FIG. 6 illustrates an example system generally at 600 that includes an example computing device 602 that is representative of one or more computing systems and/or devices that may implement the various techniques described herein. This is illustrated through the inclusion of the sequencing data processor 110. The computing device 602 may be, for example, a server of a service provider, a device associated with a client (e.g., a client device), an on-chip system, and/or any other suitable computing device or computing system.

The example computing device 602, as illustrated, includes a processing system 604, one or more computer-readable media 606, and one or more I/O interfaces 608 that are communicatively coupled, one to another. Although not shown, the computing device 602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 604 is representative of functionality to perform one or more operations using hardware. Accordingly, the processing system 604 is illustrated as including hardware elements 610 that may be configured as processors, functional blocks, and so forth. This may include implementation in hardware as an application-specific integrated circuit or other logic device formed using one or more semiconductors. The hardware elements 610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 606 is illustrated as including memory/storage 612. The memory/storage 612 represents memory/storage capacity associated with one or more computer-readable media. The memory/storage 612 may include volatile media (such as random-access memory (RAM)) and/or nonvolatile media (such as read-only memory (ROM), Flash memory, optical disks, magnetic disks, and so forth). The memory/storage 612 may include fixed media (e.g., RAM, ROM, a fixed hard drive, and so on) as well as removable media (e.g., flash memory, a removable hard drive, an optical disc, and so forth). The computer-readable media 606 may be configured in a variety of other ways as further described below.

The one or more I/O interfaces 608 are representative of functionality to allow a user to enter commands and information to the computing device 602 and also to allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, a tactile-response device, and so forth. Thus, the computing device 602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on the hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 602. By way of example, and not limitation, computer-readable media may include “computer-readable storage media” and “computer-readable signal media.” “Computer-readable storage media” may refer to media and/or devices that enable persistent and/or non-transitory storage of information in contrast to mere signal transmission, carrier waves, or signals per se. Thus, computer-readable storage media refers to non-signal-bearing media. The computer-readable storage media include hardware such as volatile and non-volatile, removable and non-removable media, and/or storage devices implemented in a method or technology suitable for storage of information such as computer-readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage devices, tangible media, or articles of manufacture suitable to store the desired information and which may be accessed by a computer.

“Computer-readable signal media” may refer to a signal-bearing medium that is configured to transmit instructions to the hardware of the computing device 602, such as via a network. Signal media typically may embody computer-readable instructions, data structures, program modules, or other data in a modulated data signal, such as carrier waves, data signals, or other transport mechanisms. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, the hardware elements 610 and the computer-readable media 606 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by the hardware elements 610. The computing device 602 may be configured to implement particular instructions and/or functions corresponding to the software and/or hardware modules. Accordingly, implementation of a module that is executable by the computing device 602 as software may be achieved at least partially in hardware, e.g., through use of computer-readable storage media and/or the hardware elements 610 of the processing system 604. The instructions and/or functions may be executable/operable by one or more articles of manufacture (for example, one or more computing devices 602 and/or processing systems 604) to implement techniques, modules, and examples described herein.

The techniques described herein may be supported by various configurations of the computing device 602 and are not limited to the specific examples of the techniques described herein. This functionality may also be implemented all or in part through use of a distributed system, such as over a “cloud” 614 via a platform 616 as described below.

The cloud 614 includes and/or is representative of a platform 616 for resources 618, which are depicted including the sequencing data processor 110. The platform 616 abstracts underlying functionality of hardware (e.g., servers) and software resources of the cloud 614. The resources 618 may include applications and/or data that can be utilized while computer processing is executed on servers that are remote from the computing device 602. Resources 618 can also include services provided over the Internet and/or through a subscriber network, such as a cellular or Wi-Fi network.

The platform 616 may abstract resources and functions to connect the computing device 602 with other computing devices. The platform 616 may also serve to abstract scaling of resources to provide a corresponding level of scale to encountered demand for the resources 618 that are implemented via the platform 616. Accordingly, in an interconnected device example, implementation of functionality described herein may be distributed throughout the system 600. For example, the functionality may be implemented in part on the computing device 602 as well as via the platform 616 that abstracts the functionality of the cloud 614.

### Conclusion

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A method for direct template indexing, comprising:
capping capture probes of a spatial indexing substrate having a plurality of spatial locations within a capture region, each of the plurality of spatial locations having a unique spatial barcode for the capture probes therein;
applying a biological sample to the capture region;
permeabilizing the applied biological sample to release nucleic acids from the biological sample;
annealing the released nucleic acids to the capture probes while preserving a spatial organization of the nucleic acids in the biological sample;
extending the annealed nucleic acids using the capture probes as templates, the extending incorporating the unique spatial barcode of an annealed capture probe into a given nucleic acid;
eluting the extended nucleic acids from the capture probes; and
regenerating the spatial indexing substrate for reuse with a different biological sample.

2. The method of claim 1, further comprising:
generating complementary deoxyribonucleic acid (cDNA) from the eluted extended nucleic acids such that a given molecule of cDNA includes the unique spatial barcode of a corresponding molecule of the eluted extended nucleic acids; and
sequencing the cDNA to generate spatially barcoded sequencing data.

3. The method of claim 2, further comprising:
generating a biological image by imaging the biological sample on the spatial indexing substrate prior to permeabilizing the biological sample; and
generating a spatial gene expression map based on the spatially barcoded sequencing data and the biological image.

4. The method of claim 3, wherein generating the spatial gene expression map comprises:
extracting spatial barcodes from the spatially barcoded sequencing data;
mapping the extracted spatial barcodes to known locations of the capture region; and
correlating gene expression data with morphological features identified in the biological image based on the extracted spatial barcodes.

5. The method of claim 1, further comprising:
applying the different biological sample to the capture region;
permeabilizing the applied different biological sample to release different nucleic acids from the different biological sample;
annealing the released different nucleic acids to the capture probes;
extending the annealed different nucleic acids using the capture probes as templates, the extending incorporating the unique spatial barcode of a corresponding spatial location into a given different nucleic acid;
eluting the extended different nucleic acids from the capture probes; and
regenerating the spatial indexing substrate for reuse.

6. The method of claim 1, wherein capping the capture probes comprises adding a chain terminator to a 3′ end of each capture probe.

7. The method of claim 1, wherein extending the annealed nucleic acids comprises using a DNA polymerase enzyme that lacks RNA-dependent DNA polymerase activity.

8. The method of claim 1, wherein eluting the extended nucleic acids comprises washing the spatial indexing substrate with a low ionic strength buffer.

9. The method of claim 1, wherein regenerating the spatial indexing substrate comprises washing the spatial indexing substrate with an alkaline solution to remove residual nucleic acids and the permeabilized biological sample.

10. A system for spatial sequencing via direct template indexing, comprising:
a spatial indexing substrate comprising:
a substrate surface;
a plurality of spatial locations on the substrate surface; and
a plurality of capture probes attached to the substrate surface at each of the plurality of spatial locations, each of the plurality of capture probes capped with a chain terminator on a 3′ end and having a spatial barcode that is specific to a corresponding spatial location; and
a sequencing data processor executing instructions stored in a non-transitory computer-readable storage medium to perform operations comprising:
receiving spatially indexed sequencing data generated from complementary deoxyribonucleic acid (cDNA) of spatially indexed messenger ribonucleic acid (mRNA), each molecule of the spatially indexed mRNA including an mRNA molecule released from a tissue sample onto the spatial indexing substrate and extended to include the spatial barcode using one of the plurality of capture probes as a template; and
generating a spatial gene expression map based on the spatially indexed sequencing data and an image of the tissue sample on the spatial indexing substrate.

11. The system of claim 10, wherein each of the plurality of capture probes comprises, from 5′ to 3′:
an anchor sequence that attaches to the substrate surface;
an amplification target sequence designed for primer binding during reverse transcription of the spatially indexed mRNA to the cDNA;
the spatial barcode;
a unique molecular identifier (UMI) sequence;
a capture sequence configured to anneal to a target nucleic acid sequence; and
the chain terminator.

12. The system of claim 11, wherein the capture sequence is a polythymidine sequence, and the target nucleic acid sequence is a polyadenylation sequence.

13. The system of claim 10, wherein the spatial indexing substrate is used with a plurality of different tissue samples during separate spatial sequencing experiments via a regeneration process performed after the spatially indexed mRNA is eluted from the plurality of capture probes.

14. The system of claim 10, wherein the mRNA molecule released from the tissue sample onto the spatial indexing substrate is extended to include the spatial barcode using a polymerase enzyme that lacks an RNA-dependent DNA polymerase activity.

15. A method for spatial sequencing via direct template indexing, comprising:
applying a tissue sample to a capture region of a spatial indexing substrate, the spatial indexing substrate having a plurality of spatial locations, each spatial location having capture probes with a spatial barcode that is specific to the spatial location and a 3′ chain terminator;
permeabilizing the applied tissue sample to release messenger ribonucleic acid (mRNA) from the applied tissue sample to the capture probes while preserving a spatial organization of the mRNA in the applied tissue sample;
annealing the released mRNA to the capture probes;
extending the annealed mRNA using a polymerase enzyme that lacks RNA-dependent deoxyribonucleic acid (DNA) polymerase activity and using the capture probes as templates, the extending producing spatially indexed mRNA encoding the spatial barcode that is specific to the spatial location of an annealed capture probe;
eluting the spatially indexed mRNA from the capture probes;
generating complementary DNA (cDNA) from the spatially indexed mRNA such that a given molecule of cDNA includes the spatial barcode of a corresponding molecule of the spatially indexed mRNA;
sequencing the cDNA to generate spatially barcoded sequencing data; and
regenerating the spatial indexing substrate by washing the spatial indexing substrate with an alkaline solution.

16. The method of claim 15, wherein the 3′ chain terminator is a dideoxynucleotide.

17. The method of claim 15, further comprising generating a tissue image by imaging the applied tissue sample on the spatial indexing substrate prior to permeabilizing the applied tissue sample.

18. The method of claim 17, further comprising generating a spatial gene expression map based on the spatially barcoded sequencing data and the tissue image.

19. The method of claim 15, wherein each capture probe further comprises a unique molecular identifier (UMI) sequence that is incorporated into the spatially indexed mRNA during the extending.

20. The method of claim 19, further comprising using the UMI sequence to identify and remove duplicate reads in the spatially barcoded sequencing data.
